(19) United States
(12) Patent Application Publication
(10) Pub. No.: US 2016/0304546 A1
(43) Pub. Date: Oct. 20, 2016

(54) COMPOUNDS AND METHODS FOR THE TREATMENT OF CANCER

(71) Applicants: John C.Jr AMEDIO, Franklin, MA (US); SOLASIA PHARMA K.K., Tokyo (JP)

(72) Inventor: John C. Amedio, Jr., Franklin, MA (US)

(73) Assignee: SOLASIA PHARMA K.K., Tokyo (JP)

(21) Appl. No.: 15/101,571

(22) PCT Filed: Dec. 5, 2014
(86) PCT No.: PCT/US14/68859

§ 371 Date: Jun. 3, 2016

Related U.S. Application Data
(60) Provisional application No. 61/912,044, filed on Dec. 5, 2013.

(51) Int. Cl. C07F 9/72; A61K 45/06; A61K 9/00; A61K 31/285

(52) U.S. Cl. CPC C07F 9/72; A61K 31/285; A61K 45/06; A61K 9/0019; C07B 2200/13

(57) ABSTRACT

The present invention relates generally to the field of anti-cancer therapy. More particularly, it provides novel crystalline forms of organic arsenic compounds and methods for their use in treating cancers such as leukemia and solid tumors. Specifically, a crystalline form of darinaparsin, wherein the crystalline form has a melting point in the range of about 190-200 deg C.

### FIELD OF THE INVENTION

The present invention relates generally to the field of anti-cancer therapy. More particularly, it provides novel crystalline forms of organic arsenic compounds and methods for their use in treating cancers such as leukemia and solid tumors.

### BACKGROUND OF THE INVENTION

Despite progress in leukemia therapy, most adult patients with leukemia still die from disease progression. Arsenic trioxide, an inorganic compound, has been approved for the treatment of patients with relapsed or refractory acute promyelocytic leukemia (APL) and is being evaluated as therapy for other leukemia types. Preliminary data from China and the recent experience in the U.S., however, suggest a role for arsenic trioxide in the other hematologic cancers as well. Consequently, the effectiveness of arsenic trioxide as an anti-leukemic agent is currently being investigated in many types of leukemia. While some leukemia types are responsive to arsenic trioxide, issues with systemic toxicity have been reported (Soignet et al., 1999; Wiernik et al., 1999; Geissler et al., 1999; Rousselot et al., 1999).

An organic arsenical (OA) manufactured for human use, melarsoprol, has been evaluated for antileukemic activity (WO9924029, EP1002537). Unfortunately, this compound is excessively toxic to patients at concentrations used for the treatment of trypanosomiasis. Another OA, darinaparsin, has shown promise as a potential new therapy for leukemia and other hyperproliferative disorders.

The properties of the solid state are critical factors that determine the choice of an appropriate salt form of a drug molecule. A given salt form may exist as different solid phases which may arise during crystallization or pharmaceutical processing. Such solid forms include polymorphs, for example. Polymorphism is the ability of a substance to exist as two or more crystalline phases that have different crystal lattices. Polymorphs have different physico-chemical properties such as crystal packing, bulk thermodynamic, spectroscopic, kinetic, surface and mechanical properties; see pages 1-33 of D. J. W. Grant, “Theory and origin of polymorphism” in: Polymorphism in Pharmaceutical Solids, ed. H.G. Brittain, Marcel Dekker, New York, 1999, incorporated herein by reference in its entirety. Different solid phases may confer different properties and characteristics to the drug molecule such as processability, stability, melting point, solubility, and shelf life which in turn may affect in vivo pharmacology, such as therapeutic efficacy, toxicity and bioavailability. Given these property differences, new crystal forms of darinaparsin would be of potential use in preparing pharmaceutical formulations.

### SUMMARY OF THE INVENTION

The present invention provides novel crystalline forms of organic arsenical compounds having anti-cancer properties. In certain embodiments, the present invention provides a crystalline form of a compound having a structure of formula (I) (darinaparsin)

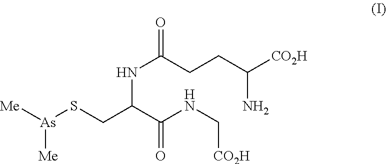

wherein the crystalline form has a melting point greater than or equal to 185° C. In some embodiments, the crystalline form has a melting point in the range of about 185-200° C. In some embodiments, the crystalline form has a melting point in the range of about 187-200° C., e.g., 187-197° C. In some embodiments, the crystalline form has a melting point in the range of about 190-200° C. In other embodiments, the invention provides a crystalline form of a compound having a structure of formula (I) (darinaparsin) wherein the crystal form has an X-ray-powder diffraction pattern comprising characteristic peaks, expressed in terms of 2θ, at one or more of the following angles: about 16.6°, about 17.4°, about 21.4° and about 25.2°.

In some embodiments, the X-ray powder diffraction pattern of the crystalline form of a compound having a structure of formula (I) (darinaparsin) also has characteristic peaks, expressed in terms of 2θ, at one or more of the following angles: about 14.4°, about 19.3°, about 22.0°, about 23.3° and about 25.0°. In some embodiments, the X-ray powder diffraction pattern of the crystalline form of a compound having a structure of formula (I) (darinaparsin) has characteristic peaks substantially as shown in FIG. 2. In some embodiments, the crystalline form of a compound having a structure of formula (I) (darinaparsin) also has a differential scanning calorimetry trace substantially as shown in FIG. 1.

In some embodiments, the invention provides a crystalline form of a compound having a structure of formula (I) (darinaparsin) wherein the crystal form has (a) a melting point greater than or equal to 185° C. and (b) an X-ray powder diffraction pattern comprising characteristic peaks, expressed in terms of 2θ, at one or more of the following angles: about 16.6°, about 17.4°, about 21.4°, and about 25.2°.

In some embodiments, the invention provides a crystalline form of a compound having a structure of formula (I) (darinaparsin) wherein the crystal form has (a) a melting point in the range of about 185-200° C. and (b) an X-ray powder diffraction pattern comprising characteristic peaks, expressed in terms of 2θ, at one or more of the following angles: about 16.6°, about 17.4°, about 21.4°, and about 25.2°.

In some embodiments, the invention provides a crystalline form of a compound having a structure of formula (I) (darinaparsin) wherein the crystal form has (a) a melting point in the range of about 190-200° C. and (b) an X-ray powder diffraction pattern comprising characteristic peaks, expressed in terms of 2θ, at one or more of the following angles: about 16.6°, about 17.4°, about 21.4°, and about 25.2°.

In some embodiments, the invention provides a method for the preparation of a pharmaceutical composition, such as an aqueous solution having a pH in the range of 4 to 7, which comprises dissolving a crystalline preparation of a compound having a structure of formula (I) as described herein in water (e.g., water for injection); and optionally adjusting the pH.

In some embodiments, the invention provides a method for the preparation of a lyophilisate, which comprises preparing an aqueous solution of a compound having a structure of formula (I) and lyophilizing the aqueous solution.

Other objects, features, and advantages of the present invention will become apparent from the following detailed description. It should be understood, however, that the detailed description and the specific examples are given by way of illustration only, since various changes and modifications within the spirit and scope of the invention will become apparent to those skilled in the art from this detailed description.

### BRIEF DESCRIPTION OF THE DRAWINGS

FIG. 1 depicts a differential scanning calorimetry (DSC) thermogram for the crystalline preparation of a compound of formula (I) according to the protocol of Example 1, which was recorded using a TA Instrument 2920 differential scanning calorimeter; 25-250° C. at 10° C./min.

FIG. 2 depicts a powder X-ray diffraction (PXRD) pattern for the crystalline preparation of a compound of formula (I) according to the protocol of Example 1, which was recorded using a Shimadzu XRD-6000 X-ray powder diffractometer in the 2θ geometry; scanning angles 2.5°-40° 2θ.

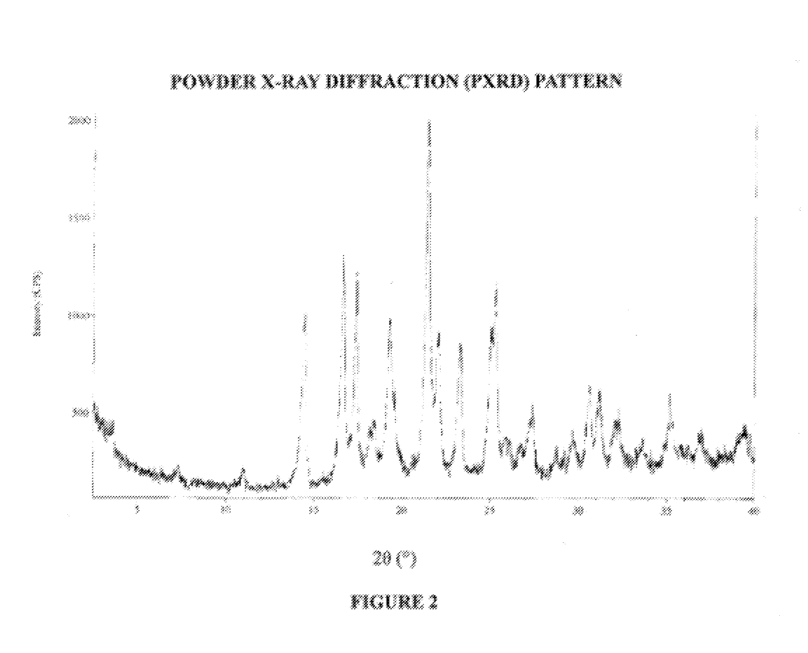

### DETAILED DESCRIPTION OF THE INVENTION

In certain embodiments of the present invention, a crystalline form of darinaparsin or a pharmaceutically acceptable salt thereof has a melting point greater than or equal to 185° C., e.g., in the range of 185-200° C. or even in the range of 187-197° C.

In certain embodiments of the present invention, a crystalline form of darinaparsin or a pharmaceutically acceptable salt thereof has a melting point in the range of 190-200° C., e.g., in the range of 190-198° C. or even in the range of 191-196° C.

The invention further provides pharmaceutical compositions including using a crystalline form of a compound having a structure of formula (I) or a pharmaceutically acceptable salt thereof, and a pharmaceutically acceptable diluent or carrier. In certain embodiments, the pharmaceutical composition is an aqueous solution that has a pH greater than about 4 or even greater than about 5, in some embodiments in the range from about 4 to about 8; in other embodiments from about 5 to about 8, in some embodiments in the range from about 4 to about 7, or in some embodiments about 5 to about 7.

In certain embodiments, a method for the preparation of a pharmaceutical composition is provided wherein the composition is an aqueous solution having a pH in a range described above that comprises a compound having a structure of formula (I) or a pharmaceutically acceptable salt thereof. Such a method comprises dissolving the crystalline form in water for injection and optionally adjusting the pH. The pH may be adjusted with pharmaceutically acceptable base or acid, such as sodium hydroxide or hydrochloric acid.

In certain embodiments, the invention relates to pharmaceutical compositions comprising a crystalline form of a compound having a structure of formula (I) or a pharmaceutically acceptable salt thereof, wherein the moisture content of the composition is less than about 10%, less than about 7%, less than about 5%, less than about 3%, or even less than about 2%. In certain such embodiments, the pharmaceutical composition is a lyophilisate.

In certain embodiments, the invention provides a composition of the compound of formula (I) or a pharmaceutically acceptable salt thereof in which at least 50% by weight of the compound of formula (I) in the pharmaceutical composition is present as the crystalline form as described herein, in some embodiments at least 70% , in other embodiments at least 80%, in other embodiments at least 90%, in other embodiments at least 95%, in other embodiments at least 97%, in other embodiments at least 99%, in other embodiments at least 99.5% or in other embodiments at least 99.9%.

In certain such embodiments, the lyophilisate comprising a crystalline form of a compound having a structure of formula (I) or a pharmaceutically acceptable salt thereof, is prepared by a method comprising preparing an aqueous solution of the crystalline form of a compound having a structure of formula (I) or a pharmaceutically acceptable salt thereof and lyophilizing the aqueous solution. In certain embodiments, the lyophilization is performed in less than about 72 hours, less than about 60 hours, less than about 48 hours, or even less than about 36 hours.

In certain embodiments, the lyophilization is performed by decreasing the temperature to about −30, about −35, about −40, about −45, or about −50° C. In some embodiments, lyophilization is performed by decreasing the temperature in the range from about −30° C. to about −50° C. In certain such embodiments, the temperature is decreased at a rate of about 1.0, about 0.7, about 0.5, about 0.3, or about 0.1° C./min. In some embodiments, the temperature is decreased at a rate of about 1.0 to about 0.1° C./min. In certain such embodiments, the composition is then held at a certain temperature as described herein for about 100, about 200, about 250, about 300, about 350, or about 400 minutes. In some embodiments, the composition is held at a certain temperature as described herein for about 100 to about 400 minutes.

In certain embodiments, the composition is then subjected to a vacuum such that the pressure is about 200, about 100, about 75, about 50, or about 25 Torr. In some embodiments, the composition is subjected to a vacuum such that the pressure is about 25 to about 200 Torr. In certain such embodiments, the temperature is increased to about −10, about −5, about 0, about 5 or about 10° C. In some embodiments, the temperature is increased from about −10° C. to about 10° C. In certain such embodiments, the temperature is increased at a rate of about 0.5, about 0.3, about 0.1, or about 0.05° C./min.

In some embodiments, the temperature is increased at a rate of about 0.05 to about 0.5° C./min. In certain such embodiments, the composition is held at a certain temperature and pressure as described herein for about 500, about 700, about 800, about 1000, about 1200, or about 1400 minutes. In some embodiments, the composition is held at a certain temperatures and pressure for about 500 to about 1400 minutes.

In certain embodiments, the composition is then subjected to a vacuum such that the pressure is either raised or lowered in relation to the abovementioned vacuum pressure and the pressure is about 200, about 100, about 75, about 50, or about 25 Torr. In some embodiments, the composition is subjected to a vacuum such that the pressure is raised or lowered and the pressure is about 25 to about 200 Torr. In certain such embodiments the temperature is increased to about 15, about 20, about 25, about 30 or about 35° C. In some embodiments, the temperature is increased from about 15° C. to about 35° C. In certain such embodiments, the temperature is increased at a rate of about 0.5, about 0.3, about 0.1, or about 0.05° C./min. In some embodiments, the temperature is increased at a rate of about 0.05 to about 0.5° C./min. In certain such embodiments, the composition is held at a given temperature and pressure for about 600, about 700, about 720, about 740, about 760, about 780, about 800, or about 900 minutes. In some embodiments, the composition is held at a certain temperatures and pressure for about 600 to about 900 minutes.

Another aspect of the invention provides a method for treating cancer, comprising administering a therapeutically effective amount of a crystalline form of a compound having a structure of formula (I) or a pharmaceutically acceptable salt thereof.

The invention also relates to the use of a crystalline form of a compound having a structure of formula (I) or a pharmaceutically acceptable salt thereof in the manufacture of a medicament for the treatment of cancer.

In certain embodiments, the cancer is selected from a solid tumor, such as brain, lung, liver, spleen, kidney, lymph node, small intestine, pancreas, blood cells, bone, colon, stomach, breast, endometrial, prostate, testicle, ovary, central nervous system, skin, head and neck, esophagus, or bone marrow, or a hematological cancer, such as leukemia, acute promyelocytic leukemia, lymphoma, multiple myeloma, myelodysplasia, myeloproliferative disease, or refractory leukemia. In certain such embodiments, the cancer is a leukemia selected from acute and chronic leukemia.

In certain embodiments, the cancer is a lymphoma selected from non-Hodgkin's and Hodgkin's lymphoma. In certain embodiments, the non-Hodgkin's lymphoma is selected from peripheral T-cell lymphoma (PTCL), diffuse large B-cell lymphoma, and marginal zone lymphoma. In certain embodiments, the Hodgkin's lymphoma is Hodgkin's nodular sclerosis.

Thus, in another aspect, the invention comprises a method of treating a patient with cancer comprising administering to the patient a composition comprising a crystalline form of a compound having a structure of formula (I) or a pharmaceutically acceptable salt thereof or a pharmaceutical composition as described above. In certain embodiments, the therapeutically effective amount of a compound may be 0.1-1000 mg/kg, 1-500 mg/kg, or 10-100 mg/kg. In particular embodiments, the method comprises administering the composition weekly, twice weekly, or even daily. It is 15 further contemplated that, in some embodiments, the treatment methods involve multiple administrations daily. In some embodiments, the method comprises administering the composition parenterally. In certain embodiments, the method comprises administering the compound daily such as by injection or infusion. Alternative routes and methods of administration described in the specification may also be used and the mode of administration will mainly depend on the type and location of the cancer. In certain embodiments, the method further comprises administering one or more additional agents to the patient. Exemplary additional agents include all-trans-retinoic acid, 9-cis retinoic acid, Am-80, or ascorbic acid. The use of other adjunct cancer therapies, such as chemotherapy, radiotherapy, gene therapy, hormone therapy, and other cancer therapies known in the art are also contemplated in conjunction with the methods of the present invention.

Various methods of administration are contemplated, including regional, systemic, direct administration and by perfusion. Such methods include administration by injection, oral routes, intravenous, intraarterial, intratumoral, administration to tumoral vasculature, intraperitoneal, intratracheal, intramuscular, endoscopical, intralesional, percutaneous, subcutaneous, topical, nasal, buccal, mucosal, anogenital, rectal and the like.

Definitions

The phrase “pharmaceutically acceptable” is employed herein to refer to those salts, excipients, carriers, ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive 15 the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “substantially free”, as used herein, refers to less than 5% by weight, preferably less than 2% by weight, more preferably less than 1% by weight.

A “therapeutically effective amount” of a compound or composition refers to an amount of the compound(s) or composition which, when administered as part of a desired dosage regimen (e.g., to a mammal, in some embodiments, a human) prevents the disease, for example in an individual that may be predisposed to it alleviates a symptom, ameliorates a condition, and/or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in manner 15 to improve or stabilize a subject's condition.

Cancer Treatment

The organic arsenicals of the current invention may be used to treat a variety of cancers, including all solid tumors and all hematological cancers, including leukemia, lymphoma, multiple myeloma, myelodysplasia, or myeloproliferative disorders. The OA is also useful for treatment of hematological cancers that have become refractory to other forms of treatment.

Leukemia is a malignant neoplasm of blood-forming tissues, characterized by abnormal proliferation of leukocytes and is one of the four major types of cancer. Leukemias are classified according to the type of leucocyte most prominently involved. Acute leukemias are predominantly undifferentiated cell populations and chronic leukemias have more mature cell forms (WO9924029).

The acute leukemias are divided into lymphoblastic (ALL) and non-lymphoblastic (ANLL) types and may be further subdivided by morphologic and cytochemical appearance according to the French-American-British classification or according to their type and degree of differentiation. Specific B- and T-cell, as well as myeloid cell surface markers/antigens are used in the classification too. ALL is predominantly a childhood disease while ANLL, also known as acute myeloid leukemia, is a more common acute leukemia among adults.

Chronic leukemias are divided into lymphocytic (CLL) and myeloid (CML) types. CLL is characterized by the increased number of mature lymphocytes in blood, bone marrow, and lymphoid organs. Most CLL patients have clonal expansion of lymphocytes with B cell characteristics. CLL is a disease of older persons. In CML, the granulocytic cells predominate at all stages of differentiation in blood and bone marrow, but may also affect liver, spleen, and other organs. Other malignant hematological diseases that may be treated with the OA of the current invention, include, but are not limited to: myelodysplasia, myeloproliferative diseases, lymphomas, and multiple myeloma.

Lymphoma is a malignant neoplasm of the hematopoietic and lymphoid tissues that develops from the abnormal growth and proliferation of lymphocytes. Lymphomas are divided into two main classes, Hodgkin's lymphoma and non-Hodgkin's lymphoma.

Hodgkin's lymphoma (HL) is characterized by the abnormal spread of tumor cells to lymph nodes throughout the body, and is often diagnosed through the appearance of multinucleated Reed-Sternberg cells upon histopathological examination. Examples of subtypes of HL that may be treated with the OA of the current invention include Hodgkin's nodular sclerosis, among others. The other major form of lymphoma, non-Hodgkin's lymphoma (NHL), describes a broad class of cancers that vary significantly in severity. Subclasses of NHL that may be treated with the OA include, but are not limited to, peripheral T-cell lymphoma (PTCL), diffuse large B-cell lymphoma, and marginal zone lymphoma.

Pharmaceutical Compositions

Pharmaceutical compositions are prepared by any suitable method, typically by uniformly mixing the active compound(s) with liquids or finely divided solid carriers, or both, in the desired proportions, and optionally forming the resulting mixture into a desired shape.

The preparation of a pharmaceutical composition that contains at least one organic arsenical or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as necessary required by FDA guidelines.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The organic arsenical may be combined with different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, intramuscularly, intraperitoneally, subcutaneously, subconjunctivally, intravesicularly, mucosally, intrapericardially, intraumbilically, intraocularly, orally, topically, locally, injection, infusion, continuous infusion, localized perfusion bathing 15 target cells directly, via a catheter, via a lavage, in lipid compositions (e.g., liposomes), or by other method or any combination of the foregoing as would be known to one of ordinary skill in the art (see, for example, Remington's Pharmaceutical Sciences, 18^{th} Ed. Mack Printing Company, 1990, incorporated herein by reference).

The actual dosage amount of a composition of the present invention administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% by weight of the organic arsenical compound. In other embodiments, the OA compound comprises between about 2% to about 75% of the weight of the composition, or between about 25% to about 60%, for example, and any range derivable therein. In other non-limiting examples, a dose also comprises from about 0.1 mg/kg/body weight, 0.5 mg/kg/body weight, 1 mg/kg/body weight, about 5 mg/kg/body weight, about 10 mg/kg/body weight, about 20 mg/kg/body weight, about 30 mg/kg/body weight, about 40 mg/kg/body weight, about 50 mg/kg/body weight, about 75 mg/kg/body weight, about 100 mg/kg/body weight, about 200 mg/kg/body weight, about 350 mg/kg/body weight, about 500 mg/kg/body weight, about 750 mg/kg/body weight, to about 1000 mg/kg/body weight or more per administration, and any range derivable therein. In non-limiting examples of a derivable range from the numbers listed herein, a range of about 10 mg/kg/body weight to about 100 mg/kg/body weight, etc., is administered, based on the numbers described above.

The desired dose is administered in a single dose or as divided doses administered 10 at appropriate intervals.

In any case, in some embodiments, the composition comprises one or more antioxidants to retard oxidation of one or more component. Additionally, in some embodiments, the prevention of the action of microorganisms is brought about by preservatives such as various antibacterial and antifungal agents, including, but not limited to parabens (e.g., methylparabens, propylparabens), chlorobutanol, phenol, sorbic acid, thimerosal or combinations thereof.

The organic arsenical may be formulated into a composition in a free base, neutral or salt form. Pharmaceutically acceptable salts include the salts formed with the free carboxyl groups derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising, but not limited to, water, ethanol, polyol (e.g., glycerol, propylene glycol, liquid polyethylene glycol, etc.), lipids (e.g., triglycerides, vegetable oils, liposomes) and combinations thereof. In some embodiments, the proper fluidity is maintained, for example, by the use of a coating, such as lecithin; by the maintenance of the required particle size, by dispersion in carriers such as, for example liquid polyol or lipids; by the use of surfactants such as, for example hydroxypropylcellulose; or combinations thereof such methods. In many cases, the compositions include isotonic agents, such as, for example, sugars, sodium chloride or combinations thereof.

Sterile injectable solutions are prepared by incorporating the active compounds in the required amount of the appropriate solvent with various of the other exemplary ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or 10 freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition should be stable under the conditions of manufacture and storage, and preserved against the contaminating action of microorganisms, such as bacteria and fungi. Thus, preferred compositions have a pH greater than about, preferably from about 5 to about 8, more preferably from about 5 to about 7. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

Combination Therapy

It is an aspect of this invention that darinaparsin can be used in combination with another agent or therapy method, preferably another cancer treatment. Darinaparsin may precede or follow the other agent treatment by intervals ranging from minutes to weeks. In embodiments where the other agent and expression construct are applied separately to the cell, one would generally ensure that a significant period of time did not elapse between the time of each delivery, such that the agent and expression construct would still be able to exert an advantageously combined effect on the cell. For example, in such instances, it is contemplated that one may contact the cell, tissue or organism with two, three, four or more modalities substantially simultaneously (i.e., within less than about a minute) with darinaparsin. In other aspects, one or more agents may be administered within about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, to about 48 hours or more prior to and/or after administering the organic arsenical. In certain other embodiments, an agent is administered within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20, to about 21 days prior to and/or after administering the organic arsenical. In some situations, it may be desirable to extend the time period for treatment significantly, however, where several weeks (e.g., about 1, about 2, about 3, about 4, about 5, about 6, about 7 or about 8 weeks or more) lapse between the respective administrations.

In some embodiments, various combinations are employed, the organic arsenical is “A” and the secondary agent, which can be one or more other therapeutic agents, is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the therapeutic compositions of the present invention to a patient will follow general protocols for the administration of chemotherapeutics, taking into account toxicity, if any. It is expected that the treatment cycles would be repeated as necessary. It also is contemplated that, in some embodiments, various standard therapies or adjunct cancer therapies, as well as surgical intervention, are applied in combination with the described arsenical agent. These therapies include but are not limited to chemotherapy, radiotherapy, immunotherapy, gene therapy and surgery. The section below describes some adjunct cancer therapies:

### Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, 15 nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, navelbine, farnesyl-protein transferase inhibitors, transplatinum, 5-fluorouracil, vincristine, vinblastine and methotrexate, or any analog or derivative variant of the foregoing.

In certain embodiments, darinaparsin is administered in combination with one or more other therapeutic agents selected from bortezomib, melphalan, dexamethasone, irinotecan, oxaliplatin, 5-fluorouracil, doxorubicin, and sorafenib. In certain embodiments, the other therapeutic agent is selected from bortezomib, dexamethasone, irinotecan, oxaliplatin, and sorafenib, preferably bortezomib. In certain embodiments, the other therapeutic agent is selected from bortezomib, dexamethasone, irinotecan, oxaliplatin, 5-fluorouracil, sorafenib, all-trans retinoic acid, 9-cis retinoic acid, Am-80 and ascorbic acid. In certain embodiments, the other therapeutic agent is selected from cisplatin (CDDP), carboplatin, procarbazine, mechlorethamine, cyclophosphamide, camptothecin, ifosfamide, melphalan, chlorambucil, busulfan, nitrosourea, dactinomycin, daunorubicin, doxorubicin, bleomycin, plicamycin, mitomycin, etoposide (VP16), tamoxifen, raloxifene, estrogen receptor binding agents, docetaxel, paclitaxel, gemcitabine, navelbine, farnesyl-protein transferase inhibitors, transplatinum, 5-fluorouracil, vincristine, vinblastine, and methotrexate, or any analog or derivative variant thereof. In certain such embodiments, the combination is synergistic. In certain alternative embodiments, the combination is additive.

In certain embodiments, darinaparsin is administered in combination with one or more other therapeutic agents such that the combination is synergistic. In certain such embodiments, the other therapeutic agent is bortezomib and/or oxaliplatin.

In certain embodiments, darinaparsin is administered in combination with one or 10 more other therapeutic agents such that the combination is additive. In certain such embodiments, the other therapeutic agent is dexamethasone, irinotecan, and/or sorafenib.

### Radiotherapy

Other factors that cause DNA damage and have been used extensively include 15 what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

### Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionucleotide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

### Gene Therapy

In yet another embodiment, the secondary treatment is a secondary gene therapy in which a therapeutic polynucleotide is administered before, after, or at the same time a first therapeutic agent. Delivery of the therapeutic agent in conjunction with a vector encoding a gene product will have a combined anti-hyperproliferative effect on target tissues.

### Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

### EXAMPLES

The following example is included to demonstrate an embodiment of the invention. It should be appreciated by those of skill in the art that the techniques disclosed in the example represent techniques discovered by the inventor to function well in the practice of the invention. However, those of skill in the art will, in light of the present disclosure, appreciate that many changes can be made in the specific 15 embodiments which are disclosed and still obtain a like or similar result without departing from the spirit and scope of the invention.

### Example 1

Preparation of Darinaparsin

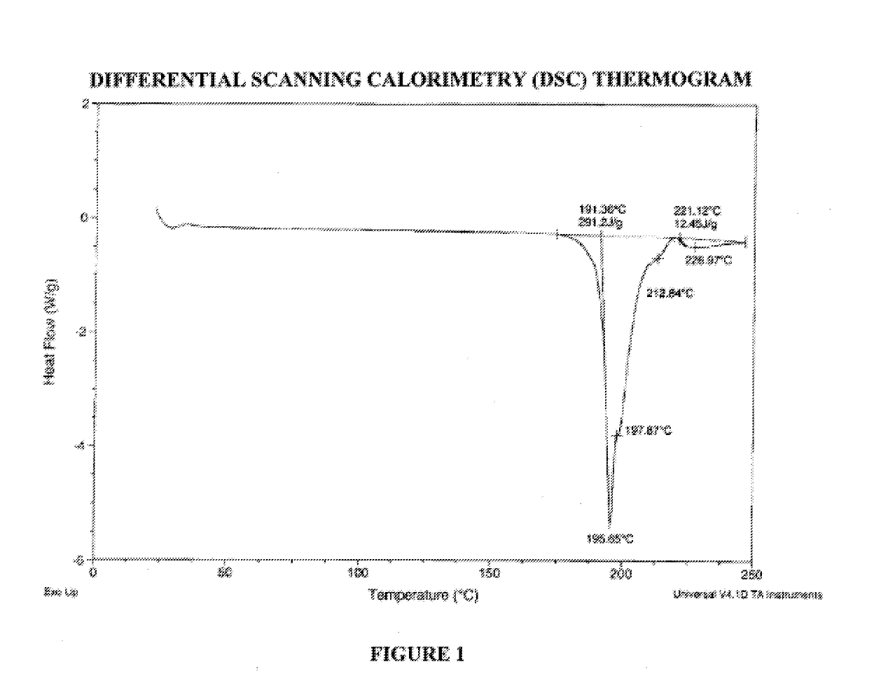

Sterile water (15.5 L) and ethyl alcohol (200 proof, 15.5 L) were charged in a reaction flask prior to the addition of L-glutathione (3.10 kg). While being stirred, the reaction mixture was cooled to 0-5° C. prior to the addition of triethylamine (1.71 L). Stirring was continued until most of the solids were dissolved and the solution was filtered. After filtration, the reaction mixture was cooled to 0-5° C. prior to the addition of chlorodimethylarsine (1.89 kg) over 115 minutes while maintaining the temperature at 0-5° C. Stirring continued at 0-5° C. for 4 hours before acetone (30.6 L) was added over 54 minutes while maintaining the temperature at 0-5° C. The suspension was stored at 0-5° C. overnight prior to filtration. The solid was collected in a filter funnel, washed successively with ethyl alcohol (200 proof, 13.5 L) and acetone (13.5 L) and dried in suction for 23 minutes. A second similar run was performed and the collected solids from both runs were combined. Ethyl alcohol (200 proof, 124 L) and the combined solids (11.08 kg) were charged in a vessel. The slurry was stirred at ambient temperature for 2 hours before filtration, washing successively with ethyl alcohol (200 proof, 27 L) and acetone (27 L) and dried in suction for 60 minutes. The resulting solid was transferred to drying trays and dried in a vacuum oven at ambient temperature for 66 hours to provide darinaparsin as a solid with the differential scanning calorimetry (DSC) thermogram of FIG. 1, with an extrapolated onset temperature at about 191.36° C. and a peak temperature at about 195.65° C.

### Equivalents

Those skilled in the art will recognize, or be able to ascertain using no more than routine experimentation, numerous equivalents to the compounds and methods of use thereof described herein. Such equivalents are considered to be within the scope of this invention and are covered by the following claims. Those skilled in the art will also 15 recognize that all combinations of embodiments described herein are within the scope of the invention.

1. A crystalline form of darinaparsin, wherein the crystalline form has a melting point in the range of about 190-200° C.

2. The crystalline form of claim 1, wherein the melting point is in the range of about 190-198° C.

3. The crystalline form of claim 1, wherein the melting point is in the range of about 191-196° C.

4. The crystalline form of claim 1, wherein the crystalline form has an X-ray powder diffraction pattern comprising characteristic peaks, expressed in terms of 2θ, about 16.6°, about 17.4°, about 21.4°, and about 25.2°.

5. The crystalline form of claim 1, wherein the crystalline form has an X-ray powder diffraction pattern comprising characteristic peaks, expressed in terms of 2θ, at about 14.4°, about 16.6°, about 17.4°, about 19.3°, about 21.4°, about 22.0°, about 23.3°, about 25.0°, and about 25.2°.

6. A crystalline form of darinaparsin, wherein the crystalline form has an X-ray powder diffraction pattern comprising characteristic peaks, expressed in terms of 2θ, about 16.6°, about 17.4°, about 21.4°, and about 25.2°.

7. The crystalline form of claim 6, wherein the crystalline form has an X-ray powder diffraction pattern comprising peaks, expressed in terms of 2θ, at about 14.4°, about 16.6°, about 17.4°, about 19.3°, about 21.4°, about 22.0°, about 23.3°, about 25.0°, and about 25.2°.

8. A method for treating cancer, comprising administering a therapeutically effective amount of the crystalline form of claims 1 or claim 6.

9. The method of claim 8, comprising orally administering the therapeutically effective amount of the crystalline form.

10. The method of claim 8, further comprising administering one or more agents or therapies.

11. The method of claim 10, wherein the one or more agents or therapies is a chemotherapeutic agent or therapy.

12. The method of claim 11, wherein the chemotherapeutic agent is selected from cisplatin (CDDP), carboplatin, procarbazine, mechlorethamine, cyclophosphamide, camptothecin, ifosfamide, melphalan, chlorambucil, busulfan, nitrosourea, dactinomycin, daunorubicin, doxorubicin, bleomycin, plicamycin, mitomycin, etoposide (VP16), tamoxifen, raloxifene, estrogen receptor binding agents, docetaxel, paclitaxel, gemcitabine, navelbine, farnesyl-protein transferase inhibitors, transplatinum, 5-fluorouracil, vincristine, vinblastine, and methotrexate, or any analog or derivative variant thereof.

13. The method of claim 10, wherein the one or more agents or therapies is a radiation therapy selected from γ-rays, X-rays, and radioisotopes; an immunotherapeutic agent or therapy; gene therapy; or surgery.

14. (canceled)

15. The method of claim 13, wherein the immunotherapeutic agent or therapy is an antibody.

16. The method of claim 15, wherein the antibody is conjugated to a drug or toxin.

17. The method of claim 16, wherein the drug or toxin is selected from all-trans retinoic acid, 9-cis retinoic acid, Am 80 ascorbic acid, a chemotherapeutic, radionucleotide, ricin A chain, cholera toxin and pertussis toxin.

18. (canceled)

19. The method of claim 17, wherein the drug or toxin is a chemotherapeutic selected from cisplatin (CDDP), carboplatin, procarbazine, mechlorethamine, cyclophosphamide, camptothecin, ifosfamide, melphalan, chlorambucil, busulfan, nitrosourea, dactinomycin, daunorubicin, doxorubicin, bleomycin, plicamycin, mitomycin, etoposide (VP16), tamoxifen, raloxifene, estrogen receptor binding agents, docetaxel, paclitaxel, gemcitabine, navelbine, farnesyl-protein transferase inhibitors, transplatinum, 5-fluorouracil, vincristine, vinblastine, and methotrexate, or any analog or derivative variant thereof.

20. The method of claim 15, wherein the antibody targets a tumor marker selected from carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B, and p155.

21-22. (canceled)

23. The method of claim 8, wherein said cancer comprises a solid tumor.

24. The method of claim 8, wherein said cancer is brain, lung, liver, spleen, kidney, lymph node, small intestine, pancreas, blood cells, bone, colon, stomach, breast, endometrium, prostate, testicle, ovary, central nervous system, skin, head and neck, esophagus, or bone marrow cancer.

25. The method of claim 8, wherein said cancer is a hematological cancer.

26. The method of claim 8, wherein said cancer is leukemia, lymphoma, multiple myeloma, myelodysplasia, myeloproliferative disease, or refractory leukemia.

27. The method of claim 8, wherein said cancer is acute promyelocytic leukemia, non-Hodgkin's lymphoma, or Hodgkin's lymphoma.

28. The method of claim 27, wherein said non-Hodgkin's lymphoma is selected from peripheral T-cell lymphoma (PTCL), diffuse large B-cell lymphoma, and marginal zone lymphoma.

29. The method of claim 27, wherein said Hodgkin's lymphoma is Hodgkin's nodular sclerosis.

30. The method of claim 8, wherein said therapeutically effective amount is 0.1-1000 mg/kg.

31-32. (canceled)

33. The method of claim 30, wherein said therapeutically effective amount is administered daily.

34-35. (canceled)

36. A pharmaceutical composition, comprising the crystalline form of any one of claims 1 or 6, and a pharmaceutically acceptable carrier or diluent.

37. A method for the preparation of a pharmaceutical composition, wherein the composition is an aqueous solution having a pH in the range of 4 to 7, comprising dissolving the crystalline form of any one of claim 1 or 6 in water for injection; and
optionally adjusting the pH.

38. The method of claim 37, wherein adjusting the pH comprises using sodium hydroxide or hydrochloric acid.

39-41. (canceled)
